(19) United States
(12) Patent Application Publication
(10) Pub. No.: US 2020/0179621 A1
(43) Pub. Date: Jun. 11, 2020

(54) DRUG DISPENSER USING COMPRESSED GAS

(71) Applicant: CG BIO CO., LTD., Seongnam (KR)

(72) Inventors: Seong Hyeon KIM, Yongin (KR); Byoung Yun KI, Seongnam (KR); Seul Ji KIM, Paju (KR)

(21) Appl. No.: 16/614,759

(22) PCT Filed: Apr. 5, 2018
(86) PCT No.: PCT/KR2018/003999

§ 371 Date: Nov. 18, 2019

(30) Foreign Application Priority Data
May 19, 2017 (KR) 10-2017-0062159

(51) Int. Cl. A61M 11/02; A61M 35/00; B05B 9/08; B05B 12/00

(52) U.S. Cl. CPC A61M 11/02; A61M 35/003; A61M 2205/8225; B05B 9/0894; B05B 12/002; B05B 9/0833

(57) ABSTRACT

The present invention relates to a drug dispenser using a compressed gas. More particularly, the present invention relates to a drug dispenser which discharges a drug in a drug container by supplying a compressed gas into the drug container. The present invention provides a drug dispenser including: a casing to which a compressed gas container for storing a compressed gas is coupled; a compressed gas gate which is provided in the casing, and opens or closes a supply of the compressed gas from the compressed gas container; a compressed gas delivery unit which delivers the compressed gas supplied from the compressed gas gate; and a dispensing unit to which a drug container is coupled and which is formed at one side of the casing to discharge the compressed gas and at least a part of a drug stored in the drug container to a drug discharge unit by supplying the compressed gas supplied from the compressed gas delivery unit into the drug container.

### TECHNICAL FIELD

The present invention relates to a drug dispenser using a compressed gas. More particularly, the present invention relates to a drug dispenser which discharges a drug in a drug container by supplying a compressed gas into the drug container.

### BACKGROUND ART

A drug dispenser in the related art is used in a medical field to dispense a drug toward body parts. As an example, there is a drug dispenser for applying a drug onto a surgical site or a diseased part.

Korean Patent Application Laid-Open No. 10-2013-0060589 (published on Jun. 10, 2013) related to the drug dispenser discloses a drug dispenser for discharging a drug in a drug storage space to the outside by using an air pump.

In addition, Korean Patent Application Laid-Open No. 10-2012-0135012 (published on Dec. 12, 2012) discloses a medical drug dispenser which is provided with a compressor for producing compressed air by being controlled by a control panel and applies a drug onto a wound.

However, there is a problem in that the drug dispenser in the related art may not accurately adjust the amount of drugs to be discharged through a drug discharge port. In addition, there is a problem in that the drug dispenser in the related art may not easily change a manner of discharging the drug. In addition, the drug dispenser in the related art needs to use the compressor which is driven by being supplied with power, and as a result, there is a problem in that the drug dispenser has a complicated configuration and manufacturing costs are increased.

### DISCLOSURE

### Technical Problem

An object of the present invention is to provide a drug dispenser capable of accurately adjusting the amount of drugs to be dispensed through a discharge portion.

Another object of the present invention is to provide a drug dispenser capable of changing a manner of dispensing a drug by allowing a dispensing unit to be attachable and detachable.

Yet another object of the present invention is to provide a drug dispenser which does not require separate power because the drug dispenser discharges a drug by using pressure of a compressed gas supplied from a compressed gas container.

### Technical Solution

The present invention provides a drug dispenser including: a casing to which a compressed gas container for storing a compressed gas is coupled; a compressed gas gate which is provided in the casing, and opens or closes a supply of the compressed gas from the compressed gas container; a compressed gas delivery unit which delivers the compressed gas supplied from the compressed gas gate; and a dispensing unit to which a drug container is coupled and which is formed at one side of the casing to discharge the compressed gas and at least a part of a drug stored in the drug container to a drug discharge unit by supplying the compressed gas supplied from the compressed gas delivery unit into the drug container.

In addition, the drug dispenser may further include a gas container storage unit which is formed at one portion of the casing and stores the compressed gas container, in which a storage cap is coupled to an end portion of the gas container storage unit.

In addition, the storage cap may come into contact with a lower surface of the compressed gas container when the storage cap is coupled to the end portion of the gas container storage unit, and the compressed gas container may be coupled to the compressed gas gate as the storage cap comes into contact with the compressed gas container.

In addition, a cap hook may be provided at an end portion of the storage cap, and a storage hook, which fixes the cap hook when the storage cap is coupled, may be provided at the end portion of the gas container storage unit.

In addition, the drug dispenser may further include a gas introducing pin which extends from a container coupling surface of the compressed gas gate, in which the gas introducing pin includes an inflow path which is formed in a central portion of the gas introducing pin and through which the compressed gas is supplied, and the gas introducing pin comes into contact with a gas container closure of the compressed gas container and penetrates the gas container closure.

In addition, the drug dispenser may further include a leakproof seal which is disposed between a container coupling surface of the compressed gas gate and a gas container closure of the compressed gas container, in which the leakproof seal prevents the compressed gas from leaking to the outside when the compressed gas is supplied to the compressed gas gate.

In addition, the compressed gas gate may further include: a push button which is exposed to the outside of the casing; a compressed gas passageway which is formed in the compressed gas gate; and an opening-closing rod which is connected to the push button and intersects the compressed gas passageway, and the opening-closing rod may move to open or close the compressed gas passageway as the push button is pushed.

In addition, the opening-closing rod may further include at least one opening-closing seal which surrounds a part of a lateral side of the opening-closing rod.

In addition, the drug dispenser may further include an adjustment unit which restricts a movement distance of the opening-closing rod.

In addition, the adjustment unit may further include an adjustment rod which restricts the movement distance of the opening-closing rod by coming into contact with the opening-closing rod.

In addition, the adjustment rod may have multiple stoppers formed in a steplike arrangement at one end portion of the adjustment rod, and the movement distance of the opening-closing rod may be restricted as the opening-closing rod comes into contact with any one of the multiple stoppers.

In addition, the adjustment unit may further include: an adjustment cam which comes into contact with the adjustment rod; and a rotary dial which is connected to the adjustment cam through a rotating shaft and exposed to the outside of the casing.

In addition, the adjustment unit may further include an identification plate which is positioned between the adjustment cam and the rotary dial and has multiple identification marks, and the rotary dial may have a window through which one of the multiple identification marks is exposed to the outside in accordance with a rotational operation of the rotary dial.

In addition, the drug dispenser may further include a drug container connection unit which extends upward from a part of the dispensing unit and is connected to the drug container.

In addition, the drug dispenser may further include a joint which has an inner surface coupled to the drug container connection unit and an outer surface of a neck portion of the drug container so as to fix the drug container, in which a shape of the joint is formed in accordance with a shape of the drug container connection unit and a shape of the neck portion of the drug container.

In addition, the dispensing unit may be detachably coupled to the casing.

In addition, one of the multiple dispensing units having the drug discharge units and the drug container connection units which are different in shape may be selected and coupled to the casing.

In addition, the dispensing unit may have a rotary type rotating adjustment device, and the rotating adjustment device may adjust the amount of drugs to be discharged in accordance with a rotational operation thereof

### Advantageous Effects

The present invention is provided with the adjustment unit for controlling the operation of the compressed gas gate, and as a result, it is possible to accurately adjust the amount of drugs to be dispensed through the drug discharge unit.

In addition, the dispensing unit is attachable and detachable, and as a result, it is possible to dispense the drug in various forms by using various types of dispensing units.

In addition, the compressed gas is supplied to the compressed gas gate as the compressed gas container is coupled to the casing without requiring separate operations, and as a result, separate power is not required.

### DESCRIPTION OF DRAWINGS

FIG. 1 is a perspective view of a drug dispenser according to a first exemplary embodiment of the present invention.

FIG. 2 is an exploded perspective view of the drug dispenser according to the first exemplary embodiment of the present invention.

FIG. 3 is a view for explaining a state in which a compressed gas container is coupled in the drug dispenser according to the first exemplary embodiment of the present invention.

FIG. 4 is a view for explaining a compressed gas gate and an adjustment unit of the drug dispenser according to the first exemplary embodiment of the present invention.

FIG. 5 is an exploded perspective view for explaining the compressed gas gate and the adjustment unit of the drug dispenser according to the first exemplary embodiment of the present invention.

FIG. 6 is a view for explaining an operation of the compressed gas gate in accordance with a rotation of a rotary dial in the drug dispenser according to the first exemplary embodiment of the present invention.

FIG. 7 is a cross-sectional view for explaining a dispensing unit of the drug dispenser according to the first exemplary embodiment of the present invention.

FIG. 8 is a perspective view of a drug dispenser according to a second exemplary embodiment of the present invention.

FIG. 9 is a cross-sectional view of the drug dispenser according to the second exemplary embodiment of the present invention.

### DESCRIPTION OF MAIN REFERENCE NUMERALS OF DRAWINGS

110: Casing

120: Compressed gas gate

121: Gas introducing pin

122: Leakproof seal

123: Push button

124: Opening-closing rod

130: Gas container storage unit

132: Storage cap

140: Compressed gas delivery unit

144: Delivery hose

150: Adjustment unit

151: Rotary dial

152: Adjustment cam

153: Identification plate

154: Rotating shaft

157: Adjustment rod

160: Dispensing unit

162: Joint

164: Drug container connection unit

166: Connector

210: Casing

223: Push button

230: Gas container storage unit

232: Storage cap

260: Dispensing unit

262: Rotating adjustment device

### BEST MODE

Hereinafter, an exemplary embodiment of the present invention will be described in detail with reference to the accompanying drawings. First, in denoting reference numerals to constituent elements of the respective drawings, it should be noted that the same constituent elements will be designated by the same reference numerals, if possible, even though the constituent elements are illustrated in different drawings. Further, in the following description of the present invention, a detailed description of publicly known configurations or functions incorporated herein will be omitted when it is determined that the detailed description may make the subject matter of the present invention unclear. Further, an exemplary embodiment of the present invention will be described below, but the technical spirit of the present invention is not limited thereto and may of course be modified and variously carried out by those skilled in the art.

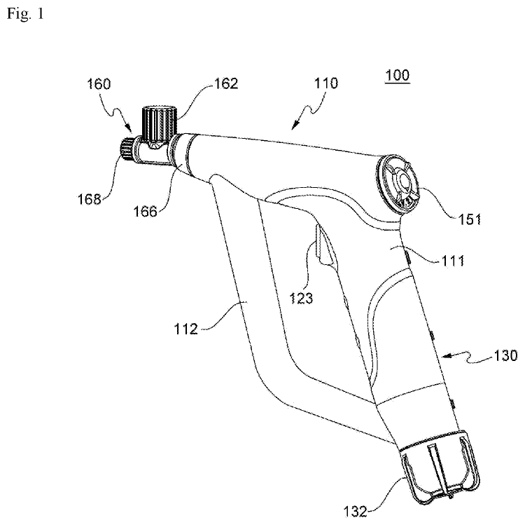

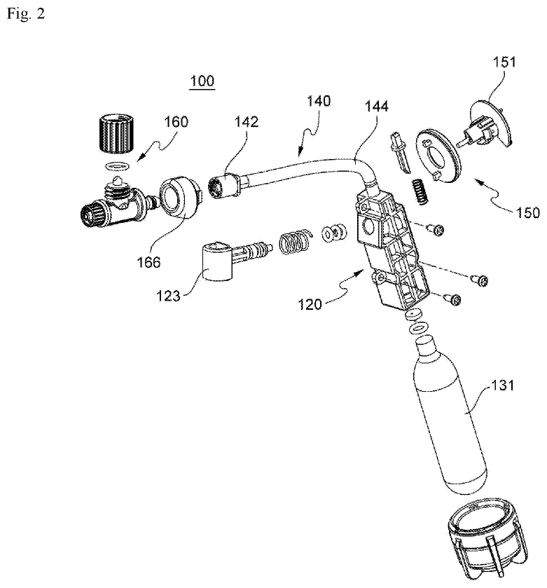

FIG. 1 is a perspective view of a drug dispenser according to a first exemplary embodiment of the present invention, and FIG. 2 is an exploded perspective view of the drug dispenser according to the first exemplary embodiment of the present invention.

As illustrated in FIGS. 1 and 2, a drug dispenser 100 according to the first exemplary embodiment of the present invention includes a casing 110, a compressed gas gate 120, a compressed gas delivery unit 140, and a dispensing unit 160. A gas container storage unit 130 for storing a compressed gas container 131 may be formed on the casing 110.

The compressed gas gate 120 and the compressed gas delivery unit 140 are provided in the casing 110, and the gas container storage unit 130 is formed at one side of the casing 110. The dispensing unit 160, which is connected to a drug container 163, is coupled to the other side of the casing.

The compressed gas container 131, which is stored in the gas container storage unit 130, is coupled to the compressed gas gate 120, the compressed gas gate 120 is connected to the compressed gas delivery unit 140, and the compressed gas delivery unit 140 is connected to the dispensing unit 160.

An operating state of the drug dispenser 100 will be described below.

A compressed gas in the compressed gas container 131 is supplied into the compressed gas gate 120. The compressed gas gate 120 determines whether to supply the compressed gas delivery unit 140 with the compressed gas supplied into the compressed gas gate 120. When the compressed gas gate 120 supplies the compressed gas to the compressed gas delivery unit 140, the compressed gas delivery unit 140 supplies the compressed gas to the dispensing unit 160. The dispensing unit 160 supplies the supplied compressed gas to the drug container 163, and the drug container 163, which has internal pressure increased by the supplied compressed gas, discharges a drug and the compressed gas to a drug discharge unit 168.

The casing 110 defines an external shape of the drug dispenser 100, and the other constituent elements are embedded in or coupled to the casing 110.

The casing 110 has the gas container storage unit 130, a handle 111, and a protection unit 112.

The handle 111 is configured to be held by one hand and to allow a push button 123 to be exposed to the outside. The protection unit 112 is structured to surround the handle 111 and protects the handle 111, thereby preventing the push button 123 from being pushed by an external object. The gas container storage unit 130 may be formed at a lower side of the handle 111.

The casing 110 may be made of a plastic material which is light in weight and easily molded.

The compressed gas delivery unit 140 is disposed in the casing 110, and may include a delivery hose 144 and a nozzle 142. The delivery hose 144 is connected to the nozzle 142 and an upper portion of the compressed gas gate 120, and the nozzle 142 is coupled to the dispensing unit 160 and a connector 166.

The compressed gas delivery unit 140 supplies the dispensing unit 160 with the compressed gas supplied from the compressed gas gate 120. The delivery hose 144 may be a hose made of a silicone material.

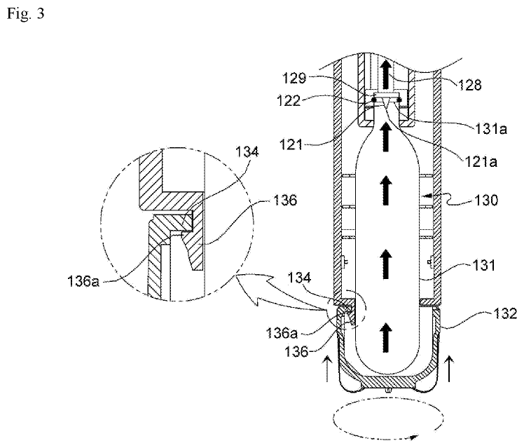

FIG. 3 is a view for explaining a state in which the compressed gas container is coupled in the drug dispenser according to the first exemplary embodiment of the present invention.

A state in which the gas container storage unit 130 and the compressed gas container 131 are coupled will be described with reference to FIG. 3.

The gas container storage unit 130 has a space into which the compressed gas container 131 may be inserted, and the gas container storage unit 130 has an opened end portion at a lower side thereof. In addition, the gas container storage unit 130 may include a storage cap 132.

The gas container storage unit 130 is provided with the opened end portion and the space into which the compressed gas container 131 may be inserted, such that the compressed gas container 131 may be stored in and withdrawn from the gas container storage unit 130.

The storage cap 132 may be coupled to the opened end portion of the gas container storage unit 130. The storage cap 132 may open and close the opened end portion of the gas container storage unit 130.

In one exemplary embodiment, the gas container storage unit 130 may have a cylindrical shape. The storage cap 132 may be coupled to the end portion of the gas container storage unit 130 by being rotated.

The gas container storage unit 130 and the storage cap 132 are configured to be coupled to each other by a threaded engagement, such that the gas container storage unit 130 and the storage cap 132 may be suitably configured to be coupled to each other by being rotated.

A length of the gas container storage unit 130 corresponds to a length of the compressed gas container 131. When the storage cap 132 is coupled to the gas container storage unit 130, the storage cap 132 pushes up the compressed gas container 131 while coming into contact with a lower surface of the compressed gas container 131, and as a result, the compressed gas container 131 is coupled to the compressed gas gate 120. Therefore, the compressed gas container 131 may be coupled by the operation of coupling the storage cap 132 to the gas container storage unit 130. A length of the gas container storage unit 130 may correspond to a length from a container coupling surface 129 of the compressed gas gate 120 to a portion where the storage cap 132 is in contact with the compressed gas container 131.

A cap hook 134 is provided at an end portion of the storage cap 132, and a storage hook 136, which is coupled to the cap hook 134 when the storage cap 132 is coupled, is provided at the end portion of the gas container storage unit 130.

The cap hook 134 is formed at an opening portion of the storage cap 132, and the storage hook 136 is formed to come into contact with a lower end of the cap hook 134 when the storage cap 132 is completely coupled to the gas container storage unit 130.

When the storage cap 132 is completely coupled to the gas container storage unit 130, the cap hook 134 passes over a hook protrusion 136a extending from a lateral side of the storage hook 136, and then engages with the storage hook 136. To decouple the storage cap 132 from the gas container storage unit 130, the cap hook 134 needs to pass over the hook protrusion 136a to start the decoupling operation.

With the aforementioned structure, there are provided an effect of allowing a user to accurately ascertain that the storage cap 132 is completely coupled, and an effect of allowing the storage cap 132 not to be easily decoupled.

The container coupling surface 129 is formed at one side of the compressed gas gate 120, and a gas introducing pin 121 extends toward the compressed gas container 131 from the container coupling surface 129.

When the compressed gas container 131 is coupled to the container coupling surface 129, the gas introducing pin 121 comes into contact with a gas container closure 131a of the compressed gas container 131 and penetrates the gas container closure 131a. The gas container closure 131a of the compressed gas container 131 is closed before the container coupling surface 129 and the compressed gas container 131 are coupled to each other, but the gas container closure 131a of the compressed gas container 131 may be torn and opened as the gas introducing pin 121 and the gas container closure 131a of the compressed gas container 131 come into contact with each other while the container coupling surface 129 and the compressed gas container 131 are coupled to each other.

An inflow path 121a through which the compressed gas is supplied may be formed in a central portion of the gas introducing pin 121. The compressed gas in the compressed gas container 131 may be supplied to a compressed gas passageway 128 of the compressed gas gate 120 through the inflow path 121a of the gas introducing pin 121 that penetrates the gas container closure 131a.

The gas introducing pin 121 may be shaped to easily tear the gas container closure 131a of the compressed gas container 131. For example, the gas introducing pin 121 may have a conical shape.

The gas container closure 131a of the compressed gas container 131 may be made of a material that is easily torn by the contact with the gas introducing pin 121.

A leakproof seal 122 is disposed between the container coupling surface 129 and the gas container closure 131a of the compressed gas container 131. The leakproof seal 122 prevents the compressed gas from leaking to the outside when the compressed gas is supplied to the compressed gas passageway 128 of the compressed gas gate 120.

When the gas container closure 131a of the compressed gas container 131 is torn by coming into contact with the gas introducing pin 121, the compressed gas in the compressed gas container 131 may not only flow into the inflow path 121a of the gas introducing pin 121, but also leak into a space between the gas introducing pin 121 and the gas container closure 131a.

The leakproof seal 122 has a ring shape surrounding the gas introducing pin 121 and is formed, without a gap, in the space between the container coupling surface 129 and the gas container closure 131a of the compressed gas container 131, thereby preventing the compressed gas, which has flown into the space between the gas introducing pin 121 and the gas container closure 131a, from leaking to the outside.

The leakproof seal 122 may be made of a material having high elastic force, and the leakproof seal 122 may be elastically deformed when the compressed gas gate 120 and the compressed gas container 131 are coupled to each other.

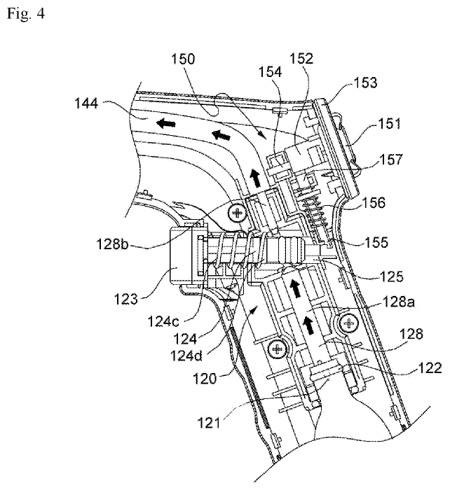

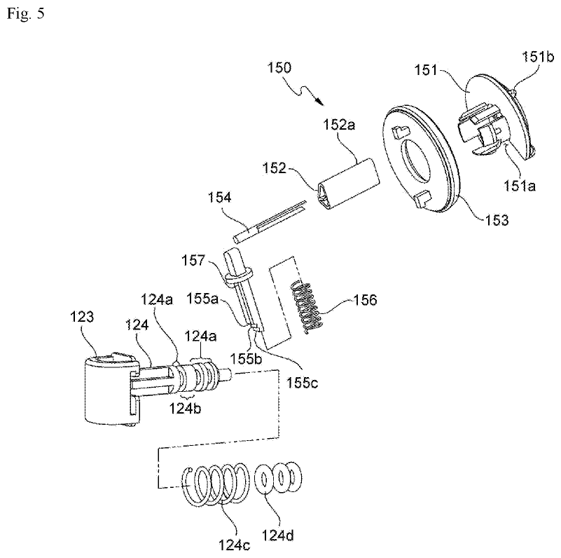

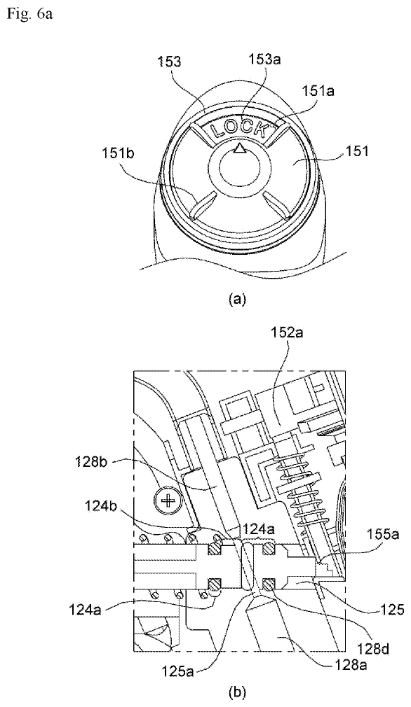

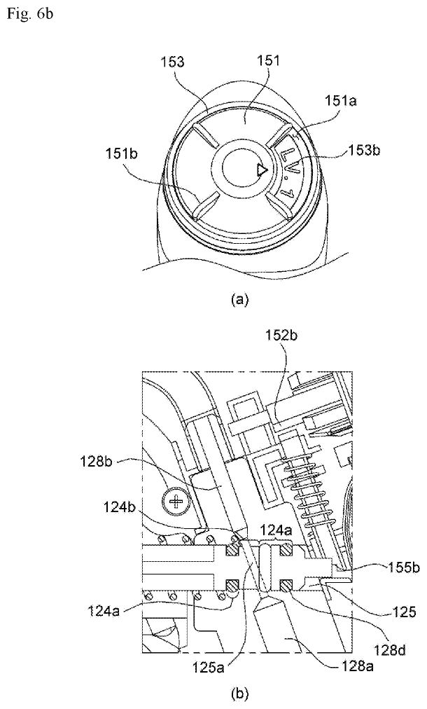

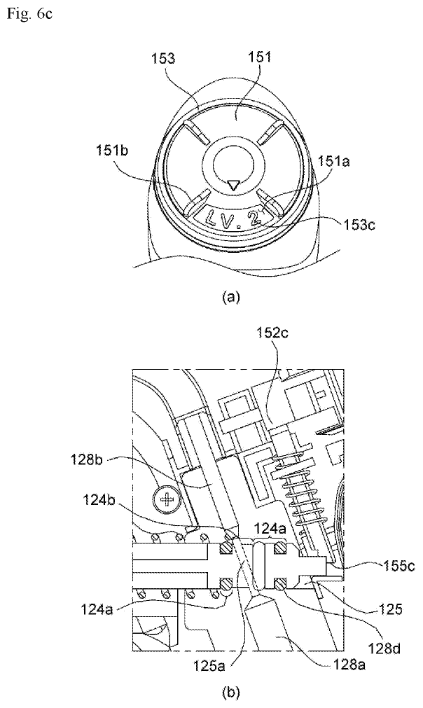

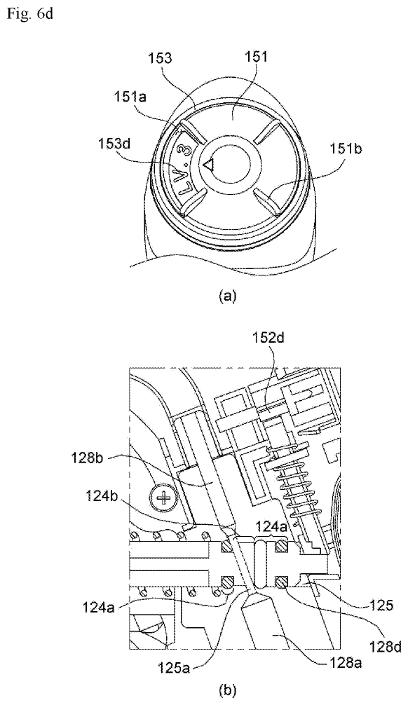

FIG. 4 is a view for explaining the compressed gas gate and an adjustment unit of the drug dispenser according to the first exemplary embodiment of the present invention, and FIG. 5 is an exploded perspective view for explaining the compressed gas gate and the adjustment unit of the drug dispenser according to the first exemplary embodiment of the present invention. In addition, FIG. 6 is a view for explaining an operation of the compressed gas gate in accordance with a rotation of a rotary dial in the drug dispenser according to the first exemplary embodiment of the present invention.

The compressed gas gate 120 and an adjustment unit 150 will be described with reference to FIGS. 4 to 6.

In one exemplary embodiment, the compressed gas gate 120 includes the compressed gas passageway 128, an opening-closing rod 124, the push button 123, and the container coupling surface 129.

The compressed gas passageway 128 is formed in the compressed gas gate 120. A lower portion of the compressed gas passageway 128 is connected to the inflow path 121a of the gas introducing pin 121, and an upper portion of the compressed gas passageway 128 is connected to the compressed gas delivery unit 140. The compressed gas, which is supplied from the inflow path 121a of the gas introducing pin 121, is supplied to the compressed gas delivery unit 140 while passing through the compressed gas passageway 128.

The compressed gas passageway 128 may intersect a rod placement portion 125. The compressed gas passageway 128 may be divided into upper and lower compressed gas passageways 128b and 128a based on a boundary where the compressed gas passageway 128 intersects the rod placement portion 125.

In one exemplary embodiment, the compressed gas passageway 128 may be formed to be narrow at a portion where the compressed gas passageway 128 is connected to the rod placement portion 125. The structure in which the compressed gas passageway 128 is narrowed may be effective in allowing the upper and lower compressed gas passageways 128b and 128a to or not to communicate with each other while the compressed gas gate 120 moves the opening-closing rod 124.

The rod placement portion 125 defines a space capable of accommodating the opening-closing rod 124. The rod placement portion 125 is provided with a connection passageway 125a which connects the upper and lower compressed gas passageways 128b and 128a, and whether the compressed gas flowing into the lower compressed gas passageway 128a is delivered to the upper compressed gas passageway 128a is determined based on an operation of opening or closing the connection passageway 125a.

The push button 123 may be exposed to the outside of the casing 110, and may be disposed at a position where the user holds the handle 111 of the casing 110 with one hand and conveniently pushes the push button 123 with his/her finger. For example, the push button 123 may be disposed at a lateral or upper side of the handle 111 of the casing 110.

The push button 123 may be sized to enable the user to apply pressure to the push button 123 with one finger. The push button 123 is connected to the opening-closing rod 124, and the opening-closing rod 124 may be moved by pushing the push button 123.

The opening-closing rod 124 is disposed in the rod placement portion 125. In one exemplary embodiment, the opening-closing rod 124 may be formed with a blocking portion 124a and a passage portion 124b, and may be provided with an opening-closing spring 124c and an opening-closing seal 124d.

The compressed gas flowing into the lower compressed gas passageway 128a needs to pass through the connection passageway 125a in the rod placement portion 125, which is connected to the upper and lower compressed gas passageways 128b and 128a, so that the compressed gas is delivered to the upper compressed gas passageway 128b, and in this case, the blocking portion 124a of the opening-closing rod 124 may block the connection passageway 125a to prevent the compressed gas from passing through the connection passageway 125a, and the passage portion 124b of the opening-closing rod 124 may not block the connection passageway 125a to allow the compressed gas to pass through the connection passageway 125a.

The opening-closing rod 124 may be moved as the push button 123 is pushed, such that the compressed gas does not pass through the connection passageway 125a when the blocking portion 124a of the opening-closing rod 124 is positioned in the connection passageway 125a as the push button 123 is pushed, while the compressed gas passes through the connection passageway 125a when the passage portion 124b of the opening-closing rod 124 is positioned in the connection passageway 125a.

If a part of the blocking portion 124a and a part of the passage portion 124b of the opening-closing rod 124 are positioned in the connection passageway 125a, the amount of compressed gas passing through the connection passageway 125a may vary in accordance with a degree to which the connection passageway 125a is blocked.

The blocking portion 124a of the opening-closing rod 124 is provided with the at least one opening-closing seal 124d which surrounds an outer circumferential surface of the opening-closing rod 124. The opening-closing seal 124d may be provided in the form of an O-ring.

The opening-closing spring 124c is provided to elastically support the opening-closing rod 124. The opening-closing spring 124c is compressed when the opening-closing rod 124 is moved as the push button 123 is pushed, and the opening-closing spring 124c is restored when the pressure is eliminated, such that positions of the push button 123 and the opening-closing rod 124 return back to the positions thereof before the push button 123 is pushed.

The adjustment unit 150 includes a rotary dial 151, an adjustment cam 152, and an adjustment rod 157.

The opening-closing rod 124 is moved as the push button 123 is pushed, a maximum movement distance of the opening-closing rod 124 is restricted by a portion which is opposite to the push button 123 and in contact with the opening-closing rod 124, and the adjustment unit 150 may adjust the maximum movement distance of the opening-closing rod 124.

The adjustment rod 157 may be movable in a longitudinal direction, and at least a part of the adjustment rod 157 may or may not come into contact with the opening-closing rod 124 in accordance with the movement of the adjustment rod 157. When the opening-closing rod 124 comes into contact with the adjustment rod 157, the maximum movement distance of the opening-closing rod 124 may be decreased.

The adjustment rod 157 may adjust the position of the opening-closing rod 124 when the push button 123 is maximally pushed while adjusting the maximum movement distance of the opening-closing rod 124, thereby determining the amount of compressed gas passing through the connection passageway 125a.

The adjustment rod 157 is provided with an adjustment spring 156. The adjustment spring 156 is compressed by external force applied to the adjustment rod 157, and the adjustment spring 156 is restored when the external force is eliminated. When the external force is eliminated, the adjustment spring 156 restores the position of the adjustment rod 157 to the position before the external force is applied.

The adjustment rod 157 is provided with multiple stoppers 155a, 155b, and 155c provided at one end portion of the adjustment rod 157 which comes into contact with the opening-closing rod 124.

The multiple stoppers 155a, 155b, and 155c are disposed in a steplike arrangement, and the opening-closing rod 124 may come into contact with one of the multiple stoppers 155a, 155b, and 155c.

The positions of the multiple stoppers 155a, 155b, and 155c, which come into contact with the opening-closing rod 124, are different from one another. Therefore, the maximum movement distance of the opening-closing rod 124 is changed as the stopper, which comes into contact with the opening-closing rod 124, is changed among the stoppers 155a, 155b, and 155c.

The adjustment rod 157 may change the stopper, which comes into contact with the opening-closing rod 124, among the stoppers 155a, 155b, and 155c while moving in the longitudinal direction, and an area of the blocking portion 124a of the opening-closing rod 124, which blocks the connection passageway 125a, is changed in accordance with the stopper, which comes into contact with the opening-closing rod 124, among the stoppers 155a, 155b, and 155c. Therefore, the amount of compressed gas passing through the connection passageway 125a may be adjusted in accordance with the arrangement of the stoppers 155a, 155b, and 155c.

In one exemplary embodiment, the adjustment cam 152 is in contact with an end portion of the adjustment rod 157 and connected to the rotary dial 151 through a rotating shaft 154.

The adjustment cam 152 is provided with multiple cam surfaces 152a, 152b, 152c, and 152d which have different heights and come into contact with the adjustment rod 157, and when the rotary dial 151, which is exposed to the outside, is rotated, the adjustment cam 152 is also rotated. When the adjustment cam 152 is rotated, the position of the adjustment rod 157 is changed in accordance with a height of the cam surface, which comes into contact with the adjustment rod 157, among the cam surfaces 152a, 152b, 152c, and 152d.

The adjustment rod 157 of which the position is changed in accordance with the rotation of the adjustment cam 152 may not come into contact with the opening-closing rod 124, and the stopper, which comes into contact with the opening-closing rod 124, may be changed among the stoppers 155a, 155b, and 155c.

The rotary dial 151 may be exposed to the outside of the casing 110, and may be rotated by the user.

As illustrated in FIG. 6, when the user rotates the rotary dial 151, the adjustment cam 152 is rotated such that the position of the adjustment rod 157 is changed, and as a result, the amount of compressed gas passing through the connection passageway 125a is adjusted as the maximum movement distance of the opening-closing rod 124 is changed.

The rotary dial 151 is provided with a window 151a through which identification marks 153a, 153b, 153c, and 153d are exposed to the outside. The user may recognize the identification marks 153a, 153b, 153c, and 153d through the window 151a.

The rotary dial 151 is provided with at least one catching portion 151b on an upper surface thereof. The user may rotate the rotary dial 151 by applying force to the catching portion 151b. The catching portion 151b provides an effect of enabling the user to more easily rotate the rotary dial 151.

An identification plate 153 is positioned between the adjustment cam 152 and the rotary dial 151, and may be provided to be fixed to the casing 110.

The identification plate 153 is provided with the multiple identification marks 153a, 153b, 153c, and 153d on an upper surface thereof. The identification marks 153a, 153b, 153c, and 153d may be letters or figures which indicate the amount of compressed gas passing through the compressed gas gate 120. When a particular identification mark is exposed to the outside among the identification marks 153a, 153b, 153c, and 153d as the rotary dial 151 is rotated, the compressed gas passes through the compressed gas gate 120 according to the amount corresponding to the exposed identification mark among the identification marks 153a, 153b, 153c, and 153d.

For example, as illustrated in FIG. 6A, when the identification mark “Lock” among the identification marks 153a, 153b, 153c, and 153d is exposed through the window 151a, the adjustment rod 157 comes into contact with the first cam surface 152a, and the maximum movement distance of the opening-closing rod 124 is restricted by the first stopper 155a, such that the opening-closing seal 124d blocks the connection passageway 125a even though the push button 123 is pushed. Therefore, the compressed gas supplied to the lower compressed gas passageway 128a does not pass through the connection passageway 125a.

In addition, as illustrated in FIG. 6B, when the identification mark “LV.1” among the identification marks 153a, 153b, 153c, and 153d is exposed through the window 151a, the adjustment rod 157 comes into contact with the second cam surface 152b, and the maximum movement distance of the opening-closing rod 124 is restricted by the second stopper 155b, such that the opening-closing seal 124d blocks only a part of the connection passageway 125a as the push button 123 is pushed. Since a part of the connection passageway 125a is blocked, the amount of compressed gas, which is supplied to the lower compressed gas passageway 128a and passes through the connection passageway 125a, is smaller than the amount of compressed gas when the connection passageway 125a is not blocked at all.

In addition, as illustrated in FIG. 6D, whether identification mark “LV.3” among the identification marks 153a, 153b, 153c, and 153d is exposed through the window 151a, the adjustment rod 157 comes into contact with the fourth cam surface 152d, and the maximum movement distance of the opening-closing rod 124 is not restricted by the stoppers 155a, 155b, and 155c, such that the opening-closing seal 124d does not block the connection passageway 125a at all when the push button 123 is pushed. Since the connection passageway 125a is not blocked at all, the amount of compressed gas, which is supplied to the lower compressed gas passageway 128a and passes through the connection passageway 125a, becomes the maximum amount.

The user may recognize the amount of compressed gas passing through the connection passageway 125a in accordance with the exposed identification mark among the identification marks 153a, 153b, 153c, and 153d.

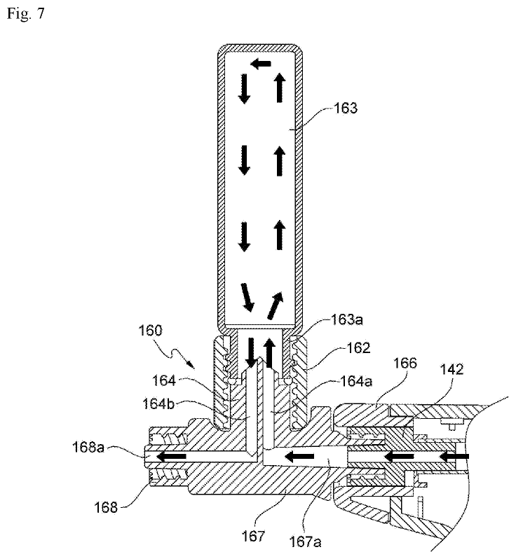

FIG. 7 is a cross-sectional view for explaining the dispensing unit of the drug dispenser according to the first exemplary embodiment of the present invention.

The dispensing unit 160 will be described with reference to FIG. 7.

The dispensing unit 160 is coupled to the casing 110 and connected to the compressed gas delivery unit 140.

The dispensing unit 160 includes a casing connection unit 167, a drug container connection unit 164, and a drug discharge unit 168.

The casing connection unit 167 may be coupled to the nozzle 142 and connected to the casing 110. The casing connection unit 167 is provided with a casing connection path 167a and delivers the compressed gas, which is supplied from the nozzle 142, to a first drug container connection path 164a. The first drug container connection path 164a supplies the delivered compressed gas into the drug container 163, and the drug and the compressed gas, which are mixed in the drug container 163, are discharged to the drug discharge unit 168 through a second drug container connection path 164b.

The drug container connection unit 164 may extend upward from a part of the dispensing unit 160 and may be in contact with a neck portion 163a of the drug container. The neck portion 163a of the drug container is formed at a lower side of the drug container 163, thereby allowing the drug to be more easily discharged.

The dispensing unit 160 may further include a joint 162 which has an inner surface coupled to the drug container connection unit 164 and an outer surface of the neck portion 163a of the drug container so as to fix the drug container 163.

The joint 162 is coupled to the drug container connection unit 164 and the neck portion 163a of the drug container, and fixes the drug container 163 so that a state in which the drug container 163 is connected to the drug container connection unit 164 is maintained. The joint 162 may have various shapes in accordance with shapes of the drug container connection unit 164 and the neck portion 163a of the drug container. Therefore, the shape of the drug container connection unit 164 and the shape of the neck portion 163a of the drug container 163 may be freely configured, and the drug container connection unit 164 and the drug container 163 may be coupled to each other by changing the shape of the joint 162.

The casing connection unit 167 of the dispensing unit 160 is detachably inserted into the nozzle 142 such that the dispensing unit 160 is fixed to the casing 110.

Since the casing connection unit 167 is attachable to and detachable from the nozzle 142, one of the multiple dispensing units 160 having different shapes may be selectively connected to the casing 110. Therefore, it is possible to change the shape of the drug discharge unit 168 and the shape of the drug container connection unit 164 only by changing the dispensing unit 160 without changing the entire drug dispenser 100. Therefore, it is possible to change a manner of discharging a drug or the drug container 163 which may be coupled.

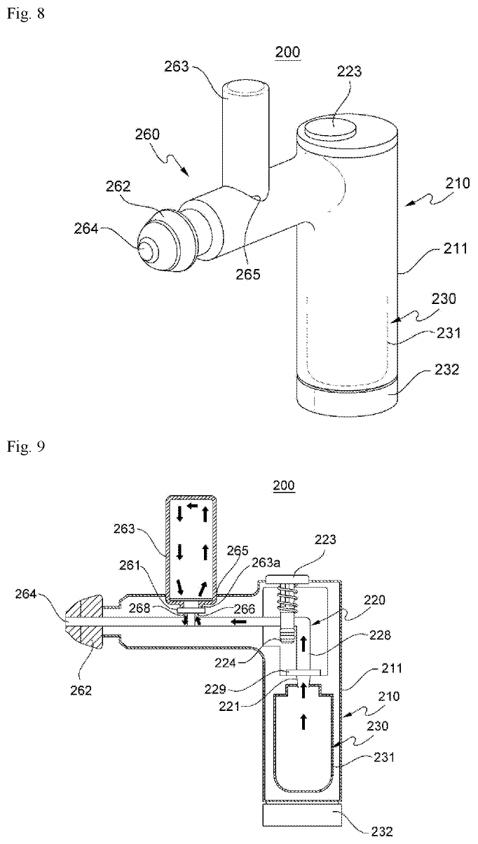

FIG. 8 is a perspective view of a drug dispenser according to a second exemplary embodiment of the present invention, and FIG. 9 is a cross-sectional view of the drug dispenser according to the second exemplary embodiment of the present invention.

A drug dispenser 200 according to the second exemplary embodiment will be described with reference to FIGS. 8 and 9.

The drug dispenser 200 may be similar to the drug dispenser 100 illustrated in FIGS. 1 and 2 except for the features to be mentioned.

The drug dispenser 200 according to the second exemplary embodiment includes a casing 210, a compressed gas gate 220, a gas container storage unit 230, and a dispensing unit 260.

The compressed gas gate 220 is provided in the casing 210, the compressed gas storage unit 230 is formed at one side of the casing 210, the dispensing unit 260 is formed at the other side of the casing 210, and the compressed gas gate 220 is connected to the dispensing unit 260.

An operating state of the drug dispenser 200 will be described below.

The compressed gas gate 220 determines whether to supply the dispensing unit 260 with the compressed gas supplied into the compressed gas gate 220. When the compressed gas gate 220 supplies the compressed gas to the dispensing unit 260, the dispensing unit 260 supplies the compressed gas to a drug container 263. The drug container 263, which has internal pressure increased by the supplied compressed gas, discharges a drug and the compressed gas to a drug discharge unit 264.

An upper portion of a compressed gas passageway 228 is connected to the dispensing unit 260. The compressed gas supplied from a gas introducing pin 221 passes through the compressed gas passageway 228 and is supplied to the dispensing unit 260.

A push button 223 may be exposed to the outside of the casing 210, and is disposed at a position where the user holds a handle 211 of the casing 210 with one hand and conveniently pushes the push button 223 with his/her finger. For example, the push button 223 may be disposed at an upper side of the handle 211 of the casing 210.

The dispensing unit 260 includes a container coupling opening 265, a drug container connection unit 261, a first connection path 266, a second connection path 268, a rotating adjustment device 262, and the drug discharge unit 264.

The container coupling opening 265 is opened toward an upper side of the casing so that a part of the drug container 263 may be inserted into the casing 210, and the drug container connection unit 261 is connected to a neck portion 263a of the inserted drug container.

When the compressed gas is supplied to the first connection path (supply path) 266 from the compressed gas passageway 228 of the compressed gas gate 220, the first connection path 266 supplies the compressed gas into the drug container 263. The drug container 263, which has internal pressure increased by the supplied compressed gas, discharges the drug and the compressed gas to the second connection path 268. The second connection path 268 delivers the supplied drug and the supplied compressed gas to the drug discharge unit 264.

According to the drug dispenser 200 according to the second exemplary embodiment, the rotating adjustment device 262 may adjust the amount of drugs to be discharged. The rotating adjustment device 262 is configured in the form of a ring surrounding the drug discharge unit 264. The rotating adjustment device 262 is configured to be rotatable by external pressure, and configured to rotate to change a cross-sectional area of the drug discharge unit 264 through which the drug may pass. The user may rotate the rotating adjustment device 262 to adjust the amount of drugs to be discharged.

The drug discharge unit 264 discharges the drug and the compressed gas supplied from the second connection path 268 to the outside.

The above description is simply given for illustratively describing the technical spirit of the present invention, and those skilled in the art to which the present invention pertains will appreciate that various modifications, changes, and substitutions are possible without departing from the essential characteristic of the present invention. Accordingly, the exemplary embodiment disclosed in the present invention and the accompanying drawings are intended not to limit but to describe the technical spirit of the present invention, and the scope of the technical spirit of the present invention is not limited by the exemplary embodiment and the accompanying drawings. The protective scope of the present invention should be construed based on the following claims, and all the technical spirit in the equivalent scope thereto should be construed as falling within the scope of the present invention.

1. A drug dispenser comprising:
a casing to which a compressed gas container for storing a compressed gas is coupled;
a compressed gas gate which is provided in the casing, and opens or closes a supply of the compressed gas from the compressed gas container;
a compressed gas delivery unit which delivers the compressed gas supplied from the compressed gas gate; and
a dispensing unit to which a drug container is coupled and which is formed at one side of the casing to discharge the compressed gas and at least a part of a drug stored in the drug container to a drug discharge unit by supplying the compressed gas supplied from the compressed gas delivery unit into the drug container.

2. The drug dispenser of claim 1, further comprising:
a gas container storage unit which is formed at one portion of the casing and stores the compressed gas container,
wherein a storage cap is coupled to an end portion of the gas container storage unit.

3. The drug dispenser of claim 2, wherein the storage cap comes into contact with a lower surface of the compressed gas container when the storage cap is coupled to the end portion of the gas container storage unit, and the compressed gas container is coupled to the compressed gas gate as the storage cap comes into contact with the compressed gas container.

4. The drug dispenser of claim 2, wherein a cap hook is provided at an end portion of the storage cap, and a storage hook, which fixes the cap hook when the storage cap is coupled, is provided at the end portion of the gas container storage unit.

5. The drug dispenser of claim 1, further comprising:
a gas introducing pin which extends from a container coupling surface of the compressed gas gate,
wherein the gas introducing pin includes an inflow path which is formed in a central portion of the gas introducing pin and through which the compressed gas is supplied, and the gas introducing pin comes into contact with a gas container closure of the compressed gas container and penetrates the gas container closure.

6. The drug dispenser of claim 1, further comprising:
a leakproof seal which is disposed between a container coupling surface of the compressed gas gate and a gas container closure of the compressed gas container,
wherein the leakproof seal prevents the compressed gas from leaking to the outside when the compressed gas is supplied to the compressed gas gate.

7. The drug dispenser of claim 1, wherein the compressed gas gate further includes: a push button which is exposed to the outside of the casing; a compressed gas passageway which is formed in the compressed gas gate; and an opening-closing rod which is connected to the push button and intersects the compressed gas passageway, and the opening-closing rod moves to open or close the compressed gas passageway as the push button is pushed.

8. The drug dispenser of claim 7, wherein the opening-closing rod further includes at least one opening-closing seal which surrounds a part of a lateral side of the opening-closing rod.

9. The drug dispenser of claim 7, further comprising:
an adjustment unit which restricts a movement distance of the opening-closing rod.

10. The drug dispenser of claim 9, wherein the adjustment unit further includes an adjustment rod which restricts the movement distance of the opening-closing rod by coming into contact with the opening-closing rod.

11. The drug dispenser of claim 10, wherein the adjustment rod has multiple stoppers formed in a steplike arrangement at one end portion of the adjustment rod, and the movement distance of the opening-closing rod is restricted as the opening-closing rod comes into contact with any one of the multiple stoppers.

12. The drug dispenser of claim 10 or 11, wherein the adjustment unit further includes: an adjustment cam which comes into contact with the adjustment rod; and a rotary dial which is connected to the adjustment cam through a rotating shaft and exposed to the outside of the casing.

13. The drug dispenser of claim 12, wherein the adjustment unit further includes an identification plate which is positioned between the adjustment cam and the rotary dial and has multiple identification marks, and the rotary dial has a window through which one of the multiple identification marks is exposed to the outside in accordance with a rotational operation of the rotary dial.

14. The drug dispenser of claim 1, further comprising:
a drug container connection unit which extends upward from a part of the dispensing unit and is connected to the drug container.

15. The drug dispenser of claim 14, further comprising:
a joint which has an inner surface coupled to the drug container connection unit and an outer surface of a neck portion of the drug container so as to fix the drug container,
wherein a shape of the joint is formed in accordance with a shape of the drug container connection unit and a shape of the neck portion of the drug container.

16. The drug dispenser of claim 1, wherein the dispensing unit is detachably coupled to the casing.

17. The drug dispenser of claim 16, wherein one of the multiple dispensing units having the drug discharge units and drug container connection units which are different in shape is selected and coupled to the casing.

18. The drug dispenser of claim 1, wherein the dispensing unit has a rotary type rotating adjustment device, and the rotating adjustment device adjusts the amount of drugs to be discharged in accordance with a rotational operation thereof
